(19) United States
(12) Patent Application Publication
(10) Pub. No.: US 2018/0021628 A1
(43) Pub. Date: Jan. 25, 2018

(54) CORE STABILITY EXERCISE MANAGEMENT SYSTEM

(71) Applicant: CRETA CO., LTD, Seoul (KR)

(72) Inventors: Kyu Jin LEE, Seoul (KR); Dae Ho NOH, Gimposi (KR)

(21) Appl. No.: 15/547,184

(22) PCT Filed: Jan. 29, 2016
(86) PCT No.: PCT/KR2016/000978

§ 371 Date: Jul. 28, 2017

(30) Foreign Application Priority Data
Jan. 29, 2015 (KR) 10-2015-0014581

(51) Int. Cl. A63B 24/00; A61B 5/22; A61B 5/00; A63B 23/02

(52) U.S. Cl. CPC A63B 24/0062; A63B 24/0087; A63B 23/0205; A61B 5/224; A61B 5/6823; A61B 5/6831; A61B 2562/0247

(57) ABSTRACT

Disclosed is a core stability exercise management system. The core stability exercise management system may comprise: at least one detection unit for detecting the force at which the abdominal muscles contract and push a pressure sensor; a reference value control unit for, if the abdominal muscles are maximally contracted, receiving the maximum value measured by the detection unit, calculating a reference value which is 1/n (n is a real number) of the maximum value, and displaying the reference value in every corresponding section on a display device; and a measurement value control unit for receiving a measurement value detected by the detection unit according to the contraction and relaxation of the abdominal muscles, and displaying the measurement value on the display device on which the reference value is displayed.

### TECHNICAL FIELD

The present invention relates to a core stability exercise management system, and more particularly, to a core stability exercise management system which displays and visually compares whether contraction and relaxation of abdominal muscles are attained to a desired degree, so as to efficiently take core stability exercise.

### BACKGROUND ART

In order for patients with low back pain to relieve pain and to improve symptoms, effect of core stability exercise is widely known. The core stability denotes a capacity for making spine stable by muscles of body (rectus abdominis muscle, external oblique abdominal muscle, internal oblique abdominal muscle, erector spinae muscles, etc.) that surround spine. The core stability exercise includes abdominal hollowing, by which abdominal muscles contract to flatten abdomen, and abdominal bracing, by which abdominal muscles contract to protrude abdomen. In such exercise, the abdominal muscles contract by using 10% or 20% of force of contracting the abdominal muscles to the maximum and the contraction maintains for a predetermined time. Then, the abdominal muscles relax and the relaxation maintains for a predetermined time. Such contraction and relaxation are repeatedly performed. However, when a user is to take such exercise by oneself, a user hardly realizes about how much the force is needed to maintain 10% or 20% of force of contracting the abdominal muscles. Accordingly, in the conventional art, a doctor or a therapist presses both sides of the abdomen of patients by using fingers and senses the pressure given to the patients so as to recommend exercises. However, such a method is a subjective judgment of a doctor or a therapist and thus, accuracy thereof decreases. As another method, electromyogram is measured to notify the force at an appropriate level. However, an electromyogram measuring device is expensive and thus, may not be easily purchased and used. Also, when electrodes are attached on the surface of the muscles for long periods of time, a signal error may be generated due to a contact failure.

### DISCLOSURE

### Technical Problem

The present invention provides a core stability exercise management system which displays and visually compares whether contraction and relaxation of abdominal muscles are attained to a desired degree, so as to efficiently take core stability exercise.

### Technical Solution

According to an aspect of the present invention, there is provided a core stability exercise management system. The core stability exercise management system may include at least one sensor for sensing force of pushing a pressure sensor after abdominal muscles contract; a reference value controller for receiving a maximum value measured in the sensor when the abdominal muscles contract to the maximum, calculating a reference value, which is 1/n (n is a real number) of the maximum value, and displaying the reference value on each corresponding section of a display device; and a measured value controller for receiving a measured value sensed from the sensor according to contraction and relaxation of the abdominal muscles and displaying the measured value on the display device where the reference value is displayed.

The reference value controller may alternately display the reference value and an initial value measured by the sensor in the state, where the abdominal muscles do not contract, on the display device. The measured value controller may display the measured value sensed in the sensor according to contraction and relaxation of the abdominal muscles on the display device, in response to sections where the reference value and the initial value are displayed.

The sensor may include: a cover filled with incompressible fluids in an inner part thereof which is sealed by an elastic side; and a pressure sensor combined to the inner part of the cover which is filled with the incompressible fluids for sensing pressure delivered through the incompressible fluids when force is applied to the cover from the outside. The pressure sensor may be combined to the elastic side within the cover or may be placed in the space where the incompressible fluids, and may transmit the sensed value to the reference value controller or the measured value controller through wired communications or wireless communications.

The sensor may further include a supporter which is made with a non-elastic material and is combined to one side of the cover for supporting the one side of the cover. The cover may include an elastic side, to which the force is applied from the outside, and at least one non-elastic side except for the side, to which the force is applied.

The core stability exercise management system may further include a notice controller for providing notice when a difference between the measured value and the reference value is over a threshold value.

The at least one sensor may be combined to a belt and thus, closely adhered to the abdominal muscles.

### Advantageous Effects

The core stability exercise management system according to an embodiment of the present invention may measure the maximum value in contracting the abdominal muscles for each person, set and display the reference value at a certain level, and display together a current degree of contraction on a screen. Accordingly, core stability exercise is easily available by oneself and the effect of the exercise may be maximized In addition, the core stability exercise management system according to an embodiment of the present invention may be realized in a low cost. Therefore, a user may take the core stability exercise easily and accurately in daily life at home or work without purchasing an expensive electromyogram measuring device.

### DESCRIPTION OF DRAWINGS

FIG. 1 is a block diagram of a core stability exercise management system according to an embodiment of the present invention;

FIG. 2 is a flowchart of a method of managing core stability exercise by using the core stability exercise management system of FIG. 1;

FIG. 3 illustrates that a sensor of FIG. 1 is equipped with a user;

FIG. 4 is a graph illustrating that a reference value controller of FIG. 1 displays a reference value on a display device;

FIG. 5 is a graph illustrating that a measured value controller of FIG. 1 displays a measured value on the display device of FIG. 4 where the reference value is displayed;

FIG. 6 is a perspective view of the sensor of FIG. 1;

FIG. 7 is a cross-sectional view of the sensor of FIG. 6;

FIG. 8 is a cross-sectional view of the sensor of FIG. 6 including a supporter;

FIG. 9 is a cross-sectional view of a sensor according to another embodiment of the present invention;

FIG. 10 is a cross-sectional view of the sensor of FIG. 6 for explaining the case where the force is applied to the sensor of FIG. 6; and

FIG. 11 is a cross-sectional view of a sensor according to another embodiment of the present invention.

### MODE FOR INVENTION

The exemplary embodiments of the present invention will be described in detail with reference to the accompanying drawings and description of the drawings so as to fully understand advantages and objectives of the present invention.

Hereinafter, embodiment of the present invention will be described more fully with reference to the accompanying drawings. In the drawings, like reference numerals denote like element.

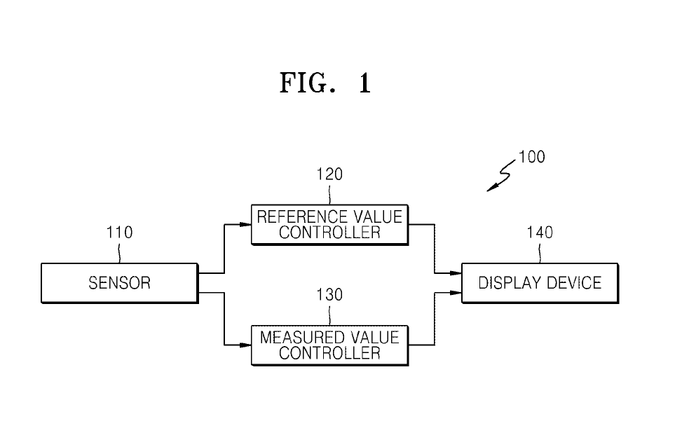

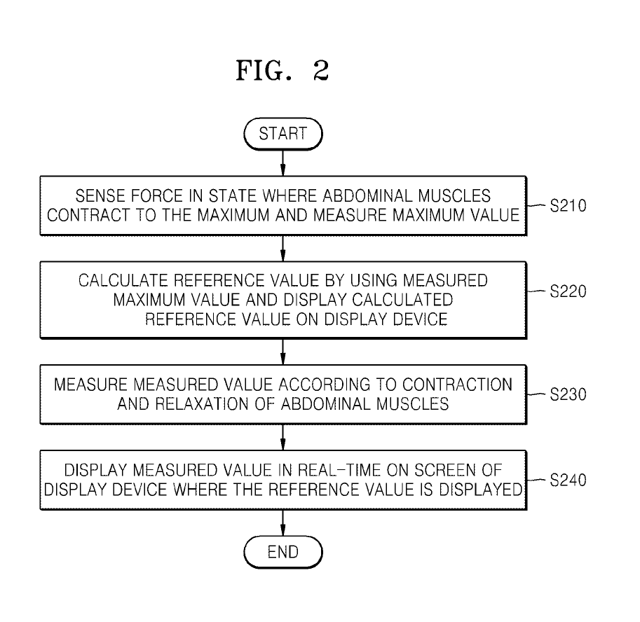

FIG. 1 is a block diagram of a core stability exercise management system 100 according to an embodiment of the present invention and FIG. 2 is a flowchart of a method of managing core stability exercise by using the core stability exercise management system 100 of FIG. 1.

Referring to FIGS. 1 and 2, the core stability exercise management system 100 may include at least one sensor 110, a reference value controller 120, a measured value controller 130, and a display device 140.

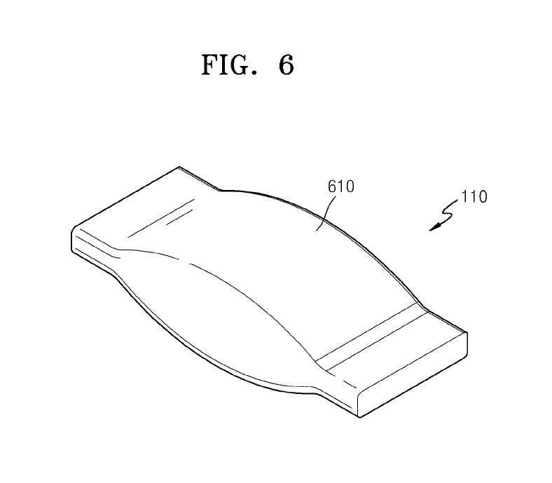

The at least one sensor 110 may sense the force applied after abdominal muscles contract. The sensor 110 may include a cover and a pressure sensor, wherein the cover is sealed with an elastic face and filled with incompressible fluids in an inner part thereof, and the pressure sensor is combined to the inner part of the cover. When force is applied from the outside of the cover, the pressure sensor may sense the delivered force through the incompressible fluids. Examples of the sensor 110 will be described in more detail with reference to FIGS. 6 through 11 below. The sensor 110 may transmit sensed results to the reference value controller 120 or to the measured value controller 130 by using wired communications or wireless communications.

When the abdominal muscles contract to the maximum, the reference value controller 120 receives a maximum value measured from the sensor 110 and calculates a reference value, which is 1/n (n is a real number) of the maximum value. Then, the reference value controller 120 may display the reference value on each corresponding section of the display device 140. The reference value represents contraction of abdominal muscles and may have, for example, 10% or 20% of the maximum value.

The measured value controller 130 receives a measured value sensed from the sensor 110 according to contraction and relaxation of the abdominal muscles and may display the measured value on a screen of the display device 140 where the reference value is displayed. The measured value is displayed on the screen of the display device 140 in real-time and a user may determine whether the force is applied to the abdominal muscles by using appropriate force and thereby, the abdominal muscles contract, by watching the reference value displayed on the display device 140 and the measured value displayed in response to the reference value on the display device 140.

The display device 140 includes a screen which may display the maximum value and the measured value and may be, for example, a smart phone, a tablet, a monitor, or a laptop.

In order to take core stability exercise, the abdominal muscles contract by using a certain degree of force (for example, 10% of force by which a user may contract the abdominal muscles to the maximum) When the abdominal muscles contract excessively, backache may be caused and when the abdominal muscles contract insufficiently, the exercise may be ineffective. Therefore, the core stability exercise management system 100 according to an embodiment of the present invention is used so that a user may identify whether appropriate force is applied to contract the abdominal muscles in real-time. Accordingly, when the abdominal muscles contract excessively, the applied force may be reduced and when the abdominal muscles contract insufficiently, more force may be applied. Thus, the core stability exercise may be effectively taken.

According to the method of taking exercise by using the core stability exercise management system 100 of FIG. 1 with reference to FIG. 2, the sensor 110 firstly senses force in the state where the abdominal muscles contract to the maximum and measures the maximum value, in operation S210. Then, the reference value controller 120 receives the measured maximum value to calculate the reference value, which is 1/n of the maximum value, and may display the calculated reference value on the display device 140, in operation S220. Then, a user contracts the abdominal muscles in the section where the reference value is displayed on the display device 140 and relaxes the abdominal muscles in the section where the reference value is not displayed. Here, such contraction and relaxation of the abdominal muscles are measured in the sensor 110 as the measured value, in operation S230. The sensor 110 transmits the sensed measured value to the measured value controller 130 and the measured value controller 130 may display the received measured value on a screen of the display device 140 where the reference value is displayed, in operation S240. Also, the measured value controller 130 may give notice when a difference between the measured value and the reference value is over a threshold value. For example, when the measured value is out of a range between a value obtained by adding the reference value to the threshold value and a value obtained by subtracting reference value from the threshold value, the measured value controller 130 gives notice so that a user may apply appropriate force to the abdomen. The notice may be a sound or may be displayed on a screen of the display device 140.

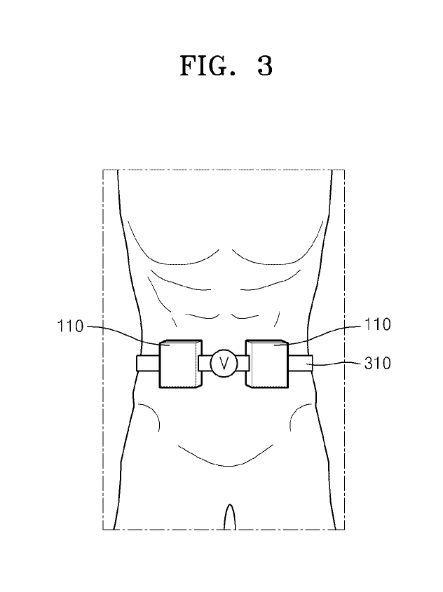

FIG. 3 illustrates that the sensor 110 of FIG. 1 is equipped with a user. Referring to FIGS. 1 through 3, the at least one sensor 110 may be combined to a belt 310 and thus closely adhered to the abdominal muscles. In FIG. 3, two sensors 110 are combined to the belt 310, however, the present invention is not limited thereto. Other numbers of sensor 110 may be combined to the sensor 110. The belt 310 may be fastened at the abdomen to press the abdomen and the sensor 110 may measure contraction of the abdominal muscles when the abdominal muscles contract while the sensor 110 presses the abdomen.

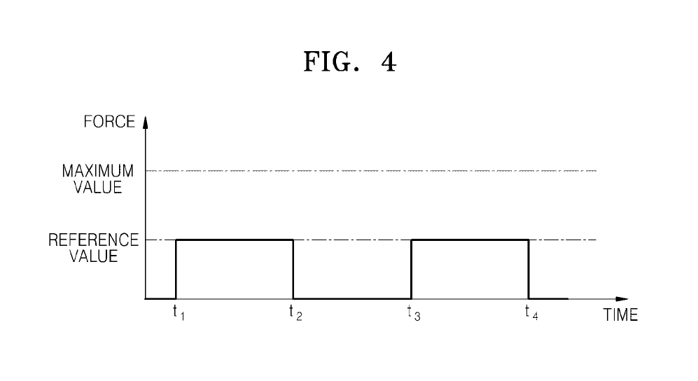

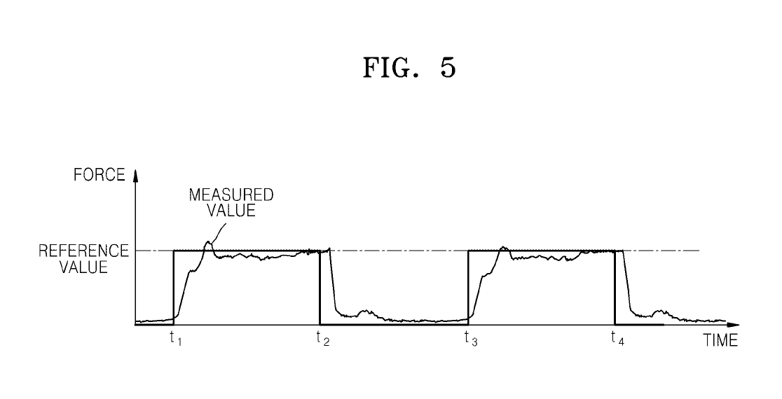

FIG. 4 is a graph illustrating that the reference value controller 120 of FIG. 1 displays the reference value on the display device 140 and FIG. 5 is a graph illustrating that the measured value controller 130 of FIG. 1 displays the measured value on the display device 140 of FIG. 4 where the reference value is displayed.

Referring to FIGS. 1 through 5, the reference value controller 120 may alternately display the reference value and an initial value measured by the sensor 110 in the state, where the abdominal muscles do not contract, on the display device 140. For example, when a user is to repeat contraction and relation of the abdominal muscles in every 10 seconds, the reference value controller 120 may alternately display the reference value and the initial value on the display device 140 in every 10 seconds. As another example, when a user is to take exercise contracting the abdominal muscles for 10 seconds and relaxing the abdominal muscles for 20 seconds, the reference value controller 120 may repeatedly display the reference value for 10 seconds and then, the initial value for 20 seconds on the display device 140. That is, the reference value controller 120 may display the reference value between t1 and t2 and between t3 and t4, and the initial value between t2 and t3.

When the reference value is determined by using such maximum value, a user actually starts taking core stability exercise after the sensor 110 is adhered to the abdomen. Here, according to contraction and relation of the abdominal muscles by the user, the measured value controller 130 displays the measured value on the display device 140 as illustrated in FIG. 5. That is, the abdominal muscles start contracting from t1 and contraction of the abdominal muscles maintains to t2. Here, the user may realize whether the force to the abdominal muscles is applied or reduced while watching the measured value displayed on the display device 140 in real-time. As such, in order for a user to easily realize the time for contracting or relaxing the abdominal muscles, the measured value controller 130 may display the measured value on the display device 140, in response to the reference value and the initial value. For example, as illustrated in FIG. 5, the reference value and the measured value may overlap each other. Also, sections of the reference value and the measured value are united while the reference value and the measured value do not overlap each other and the reference value and the measured value may be displayed on one screen. In addition, the measured value controller 130 may give notice when a difference between the measured value and the reference value is over a threshold value.

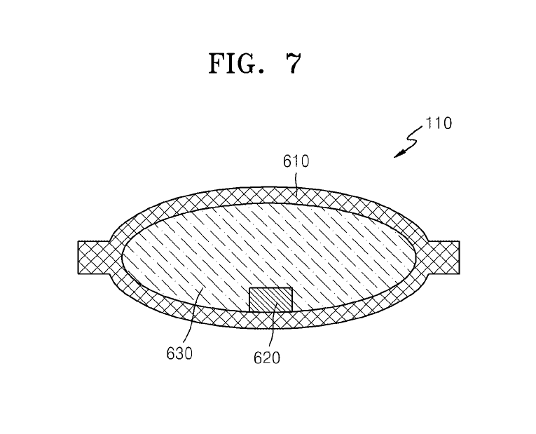

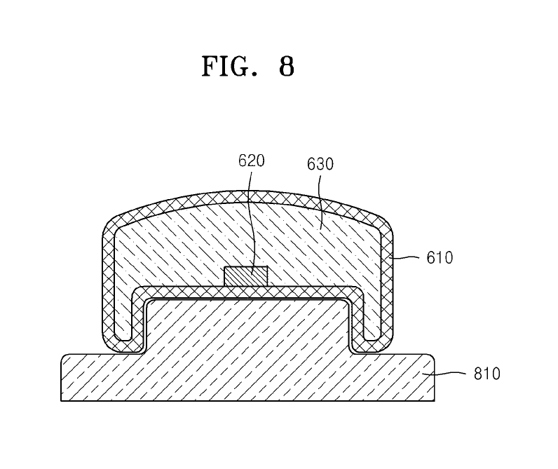

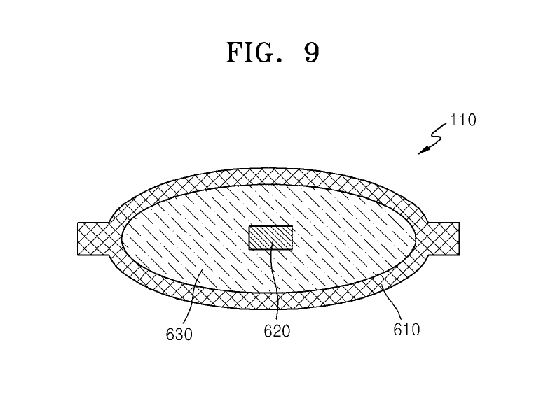

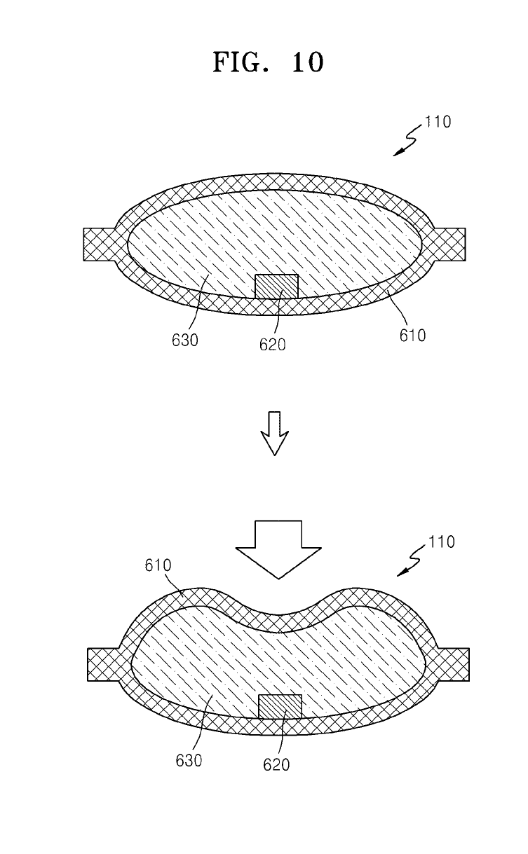

FIG. 6 is a perspective view of the sensor 110 of FIG. 1, FIG. 7 is a cross-sectional view of the sensor 110 of FIG. 6, FIG. 8 is a cross-sectional view of the sensor 110 of FIG. 6 including a supporter 810, FIG. 9 is a cross-sectional view of a sensor 110′ according to another embodiment of the present invention, and FIG. 10 is a cross-sectional view of the sensor 110 of FIG. 6 for explaining the case where the force is applied to the sensor 110 of FIG. 6.

Referring to FIGS. 1 through 10, the sensor 110 may include a cover 610, a pressure sensor 620, and incompressible fluids 630.

The cover 610 is sealed with an elastic face and the incompressible fluids 630 may be filled in the inner part of the sealed space. The pressure sensor 620 is combined to the inner part of the cover 610 where the incompressible fluids 630 is filled. When the force is applied to the cover 610 from the outside, the pressure may be sensed trough the incompressible fluids 630. Although not illustrated in FIGS. 6 through 10, a value measured in the pressure sensor 620 may be delivered to the reference value controller 120 or the measured value controller 130 as an electric signal through wired communications or wireless communications.

The sensor 110 may further include the supporter 810. The supporter 810 is made with a non-elastic material and is combined to one side of the cover 610. Thus, the supporter 810 may support the one side of the cover 610. The supporter 810 may be formed as illustrated in FIG. 8, however, the present invention is not limited thereto. The supporter 810 may have other various forms. Also, when the supporter 810 does not exist and when a surface of the cover 610, which is opposite the surface to which the force is applied, is put on a bottom or a wall and the force is applied to the surface, the force applied from the outside is all delivered to the pressure sensor 620 through the incompressible fluids 630. Accordingly, even if the supporter 810 does not exist, the sensor 110 may be used to accurately measure the force applied from the outside.

When the force is applied from the outside, the pressure sensor 620 may sense the pressure delivered through the incompressible fluids 630 and accurately sense a degree of the force applied from the outside. For example, when a cover is filled with compressible fluids such as air and the force is applied from the outside, the elastic surface of the cover is modified and the fluids is compressed. Thus, the force applied from the outside may not be accurately measured in the pressure sensor. However, when the incompressible fluids 630 is filled with the cover 610, which is made with an elastic material, as in the present invention, the force applied from the outside is delivered to the pressure sensor 620 through the incompressible fluids 630 even if the elastic cover 610 is slightly modified due to the force applied from the outside. Accordingly, the pressure sensor 620 may sensitively measure pressure from the outside.

As the pressure sensor 620 is combined to the inside of the cover 160, the pressure sensor 620 may be combined to the elastic side within the cover 610 or may be placed in the space where the incompressible fluids 630 exist. For example, as illustrated in FIGS. 6 through 8, the pressure sensor 620 may be combined to the lower surface of the cover 610. In this case, a direction of applying the force from the outside may be a lower direction from the upper part, an upper direction from the lower part, or an oblique direction. Accordingly, when the force is applied from any direction, the pressure sensor 620 may be used to measure the force. As another example, as illustrated in FIG. 9, the pressure sensor 620 may be placed in the space where the incompressible fluids 630 exist. The pressure sensor 620 according to the present invention does not need to be placed at the center, however, the pressure sensor 620 may be placed in any space where the incompressible fluids 630 exist while the pressure sensor 620 is not combined to the cover 610. Also, in this case, even if the force is applied from any direction, the pressure sensor 620 may accurately measure the delivered force. That is, if the pressure sensor 620 is able to receive the force applied from the outside through the incompressible fluids 630, the pressure sensor 620 may be placed anywhere within the cover 610.

When the force is applied from the upper direction to the lower direction from the outside, a change in the pressure of the incompressible fluids 630 within the cover 610 may be detected from the pressure sensor 620 and thus, a degree of the force applied from the outside may be detected. In the conventional art, accurate measurement may be available only when the force is applied in a vertical direction and accurate measurement may not be available for the force applied in an oblique direction. However, in the present invention, the sealed space within the cover 610 is filled with the incompressible fluids 630 and a change in the pressure of the incompressible fluids 630 is detected from the pressure sensor 620. Accordingly, a degree of the force may be accurately measured regardless of a direction of the force applied from the outside. Also, as the cover 610 is made with an elastic material, a pain of the body may not be caused or minimized, even if the cover 610 touches the body to measure the force. For example, when the intensity of the force according to contraction of the abdominal muscles by applying force to the abdomen is to be measured, the sensor 110 is combined to a waist support or a belt in an abdomen direction and then, the abdominal muscles contract by applying the force to the abdomen. Then, the elastic side of the cover 610 is also modified and thus, a pain of the abdomen may not be caused or minimized.

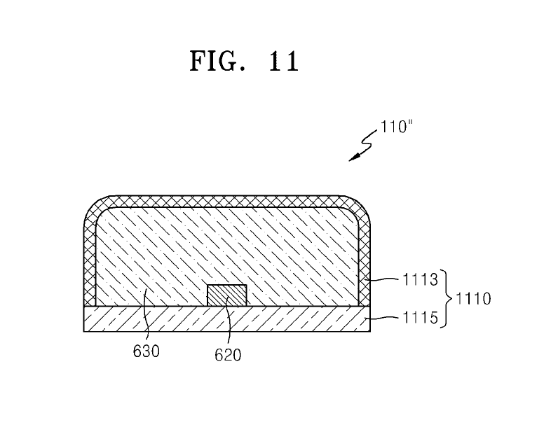

FIG. 11 is a cross-sectional view of a sensor 110″ according to another embodiment of the present invention.

Referring to FIGS. 1 through 11, the sensor 110″ of FIG. 11 may include a cover 1110, wherein the cover 1110 includes an elastic side 1113, to which the force is applied, and at least one non-elastic side 1115 from sides except for the side, to which the force is applied. Also, the cover 110 may be filled with incompressible fluids 630 and sealed. The pressure sensor 620 is combined to the inner part of the cover 1110. Accordingly, when the force is applied to the cover 1110 from the outside, the delivered pressure may be sensed trough the incompressible fluids 630. In FIG. 11, the cover 610 and the supporter 810 of FIG. 8 are combined to be formed as one cover 1110. Other elements except for the cover 1110 are similar as illustrated with reference to FIGS. 6 through 10 and thus, explanation thereof is as in that of FIGS. 6 through 10.

The sensor 110 according to the present invention is not limited to forms in FIGS. 5 through 11 and may have other various forms if the force applied from the outside may be accurately measured.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A core stability exercise management system comprising:
at least one sensor for sensing force of pushing a pressure sensor after abdominal muscles contract;
a reference value controller for receiving a maximum value measured in the sensor when the abdominal muscles contract to the maximum, calculating a reference value, which is 1/n (n is a real number) of the maximum value, and displaying the reference value on each corresponding section of a display device; and
a measured value controller for receiving a measured value sensed from the sensor according to contraction and relaxation of the abdominal muscles and displaying the measured value on the display device where the reference value is displayed.

2. The core stability exercise management system of claim 1, wherein the reference value controller alternately displays the reference value and an initial value measured by the sensor in the state, where the abdominal muscles do not contract, on the display device and the measured value controller displays the measured value sensed in the sensor according to contraction and relaxation of the abdominal muscles on the display device, in response to sections where the reference value and the initial value are displayed.

3. The core stability exercise management system of claim 1, wherein the sensor comprises:
a cover filled with incompressible fluids in an inner part thereof which is sealed by an elastic side; and
a pressure sensor combined to the inner part of the cover which is filled with the incompressible fluids for sensing pressure delivered through the incompressible fluids when force is applied to the cover from the outside.

4. The core stability exercise management system of claim 3, wherein the pressure sensor is combined to the elastic side within the cover or is placed in the space where the incompressible fluids, and transmits the sensed value to the reference value controller or the measured value controller through wired communications or wireless communications.

5. The core stability exercise management system of claim 3, wherein the sensor further comprises a supporter which is made with a non-elastic material and is combined to one side of the cover for supporting the one side of the cover.

6. The core stability exercise management system of claim 3, wherein the cover comprises an elastic side, to which the force is applied from the outside, and at least one non-elastic side except for the side, to which the force is applied.

7. The core stability exercise management system of claim 1, further comprising:
a notice controller for providing notice when a difference between the measured value and the reference value is over a threshold value.

8. The core stability exercise management system of claim 1, wherein the at least one sensor is combined to a belt and thus, closely adhered to the abdominal muscles.
